(19) United States
(12) Patent Application Publication
(10) Pub. No.: US 2025/0384953 A1
(43) Pub. Date: Dec. 18, 2025

(54) METHODS AND SYSTEMS FOR DETECTING STRUCTURAL VARIANTS

(71) Applicant: The Hong Kong University of Science and Technology, Hong Kong (CN)

(72) Inventors: Weichuan YU, Hong Kong (CN); Jiandong SHI, Hong Kong (CN); Jingyu HAO, Hong Kong (CN)

(21) Appl. No.: 19/189,396

(22) Filed: Apr. 25, 2025

Related U.S. Application Data
(60) Provisional application No. 63/659,855, filed on Jun. 14, 2024.

(51) Int. Cl. G16B 20/20; G16B 30/10; G16B 30/20; G16B 40/00

(52) U.S. Cl. CPC G16B 20/20; G16B 30/10; G16B 30/20; G16B 40/00

(57) ABSTRACT

Methods and systems for detecting structural variants (SVs) from long-read sequencing data. The method including: encoding SV signals from aligned reads in a binary alignment map (BAM) file, wherein the SV signals are encoded in a matrix form; detecting one or more candidate regions from the encoded SV signals, wherein the one or more candidate regions comprise SV signals above a pre-determined signal level; clustering the aligned reads within each of the one or more candidate regions to form one or more clusters, respectively; assembling the aligned reads within each of the one or more clusters to generate one or more contigs, respectively; aligning the one or more contigs to a respective reference sequence to detect a presence of SVs.

### CROSS-REFERENCE TO RELATED APPLICATIONS

This application claims priority to, and the benefit of, U.S. Provisional Patent Application Ser. No. 63/659,855 filed Jun. 14, 2024, the disclosure of which is incorporated by reference herein in its entirety.

### FIELD OF INVENTION

The present invention relates broadly, but not exclusively, to methods and systems for detecting structural variants.

### BACKGROUND

Structural variants (SVs) are genomic variants longer than 50 base pairs (bps). SVs are demonstrated to have a significant impact on human diversity and are closely associated with genetic diseases. While single-nucleotide variants (SNVs) and small insertions/deletions (indels) are more common, SVs collectively alter a substantial portion of the human genome. Previous research on SV calling during the era of short-read sequencing focused on methods such as read depths, discordant read pairs, split-read alignment, and local assembly. These approaches have contributed to large-scale genomics studies like the 1000 Genomes Project. However, the limited read length of short-read sequencing hampers the detection of long SVs, and challenges arise from problematic alignment, leading to potential false positive findings.

The advent of long-read sequencing technologies, such as PacBio and Nanopore, has revolutionized SV detection by enabling the acquisition of longer reads spanning thousands of bps. This advancement significantly improves the detection of SVs and facilitates more accurate analysis. Simple SVs can be categorized as balanced (e.g., inversions and translocations) or unbalanced (e.g., deletions, insertions, and duplications), where the unbalanced ones alter the length of the genome. In addition to these simple SVs, complex SVs (also known as nested SVs) have been observed in real datasets and are of particular interest. Complex SVs can be regarded as combinations of simple SVs in various orders and numbers. Specifically, translocations can be viewed as a special type of complex SV based on deletions and insertions.

To address the challenges of SV detection, numerous computational algorithms and tools have been developed. Most of these approaches focus on detecting simple SVs and can be broadly categorized into alignment-based methods and assembly-based methods.

Alignment-based methods typically involve three steps. Firstly, SV signals are captured from individual reads by utilizing compact idiosyncratic gapped alignment report (CIGAR) information from binary alignment map (BAM) files and supplementary alignment data. These signals are usually referred to as SV signatures. SV signatures are represented as {potential type, start position, length}. Secondly, SV signatures with the same potential type are clustered using thresholds or density-based methods. Within each cluster, SV signatures are synthesized based on the start position and length, resulting in potential SV calls. Finally, the quality or confidence score of each potential SV call is calculated by certain indexes, such as the number of supporting reads. By applying user-defined thresholds to the calculated scores, SV calls are finalized and reported in variant call format (VCF) files.

Alignment-based methods are popular due to their simplicity and efficiency, particularly in detecting simple SVs. However, alignment-based methods may yield false positive findings due to problematic alignment, and their pipeline determines the type of a potential SV call from the initial step, limiting the detection of complex SVs.

Assembly-based methods, on the other hand, address the limitations of alignment-based methods by first assembling reads into longer contigs and subsequently aligning these contigs to the reference genome to detect SVs. By bypassing the alignment of individual reads, assembly-based methods are less influenced by alignment results, but they are computationally expensive and not suitable for large-scale data analysis.

### SUMMARY

Embodiments of the invention provide a general SV detector using the third-generation sequencing data, comprising one or more of the following steps: encoding, detecting, clustering, assembling, aligning and finalizing. Within each candidate region, reads within a cluster are then assembled into a contig, which is mapped to a reference genome via maximum exact match. Embodiments of the invention combine the advantages of alignment-based and assembly-based methods and allow for the detection of complex SVs by deferring the assignment of SV type labels until the final alignment step. In particular, the steps include: (a) Extracting SV signals from aligned reads in BAM files; (b) Detecting regions with strong signal accumulation from reads; (c) Clustering the corresponding reads in the candidate regions; (d) Assembling the reads in one cluster into a contig; (e) Aligning the contig to the reference; and (f) Finalizing the SV calling from the alignment results from step (e). The information in step (a) can be obtained by both intra- and inter-alignment results. The detecting step (b) applies graph cut methods for detecting the strong signals. The clustering step (c) is carried out to deal with potential subtype issues. The assembly step (d) can be carried out by the wtdbg2 assembly tool.

According to one aspect, there is provided a method for detecting structural variants (SVs) from long-read sequencing data, including: encoding SV signals from aligned reads in a binary alignment map (BAM) file, wherein the SV signals are encoded in a matrix form; detecting one or more candidate regions from the encoded SV signals, wherein the one or more candidate regions comprise SV signals above a pre-determined signal level; clustering the aligned reads within each of the one or more candidate regions to form one or more clusters, respectively; assembling the aligned reads within each of the one or more clusters to generate one or more contigs, respectively; aligning the one or more contigs to a respective reference sequence to detect a presence of SVs.

The step of aligning the one or more contigs to a respective reference sequence to detect a presence of SVs may include using copmem2 to perform maximum exact match (MEM) alignment.

Subsequent to aligning the one or more contigs to a respective reference sequence to detect a presence of SVs, the method may further include filtering results of the MEM alignment to generate a SV calling, wherein the filtering is based on inversions and/or gaps in the one or more contigs.

The method may include using a wtdbg2 assembly tool to assemble the aligned reads within each of the one or more clusters to generate the one or more contigs, respectively.

The method may include using a graph cut method for detecting the one or more candidate regions from the encoded SV signals.

According to another aspect, there is provided a system for detecting structural variants (SVs) from long-read sequencing data, comprising: a processor module; and a memory module including computer program code. The memory module and the computer program code may be configured to, with the processor module, cause the system at least to: encode SV signals from aligned reads in a binary alignment map (BAM) file, wherein the SV signals are encoded in a matrix form; detect one or more candidate regions from the encoded SV signals, wherein the one or more candidate regions comprise SV signals above a pre-determined signal level; cluster the aligned reads within each of the one or more candidate regions to form one or more clusters, respectively; assemble the aligned reads within each of the one or more clusters to generate one or more contigs, respectively; align the one or more contigs to a respective reference sequence to detect a presence of SVs.

The system may be further caused to use copmem2 to perform maximum exact match (MEM) alignment for aligning the one or more contigs.

The system may be further caused to filter results of the MEM alignment to generate a SV calling, wherein the filtering is based on inversions and/or gaps in the one or more contigs.

The system may be further caused to use a wtdbg2 assembly tool to assemble the aligned reads within each of the one or more clusters to generate the one or more contigs, respectively.

The system may be further caused to use a graph cut method for detecting the one or more candidate regions from the encoded SV signals.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 is a flow chart illustrating a method for detecting structural variants (SVs) from long-read sequencing data, according to an embodiment.

FIG. 2 shows an example of a complex SV comprising deletion, duplication and inversion.

FIG. 3 is a table illustrating the performance of embodiments of the invention in detecting the SVs, compared to existing methods.

FIG. 4A is an Integrative Genomics Viewer (IGV) screenshot of a simulated DEL.

FIG. 4B is a IGV screenshot of a simulated DUP.

FIG. 5 shows the performance of candidate methods in detecting different types of complex SVs by evaluating the region and type respectively.

FIG. 6 is a flow chart illustrating a method for detecting structural variants (SVs) from long-read sequencing data, according to an embodiment.

FIG. 7 shows a schematic diagram of a computer system suitable for use in executing at least some steps of the method for detecting structural variants (SVs) from long-read sequencing data.

### DETAILED DESCRIPTION

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a conventional computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM, GPRS, 3G or 4G mobile telephone systems, as well as other wireless systems such as Bluetooth, ZigBee, Wi-Fi. The computer program when loaded and executed on such a computer effectively results in an apparatus that implements the steps of the preferred method.

The present invention may also be implemented as hardware modules. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC) or Field Programmable Gate Array (FPGA). Numerous other possibilities exist. Those skilled in the art will appreciate that the system can also be implemented as a combination of hardware and software modules.

Structural variants (SVs) have gained significant attention in genomic research due to their significance in understanding genetic diseases, gene expression, and chromosome evolution. With the advancements in long-read sequencing technologies, researchers now have the ability to obtain longer reads, thereby increasing the potential for SV detection beyond single-nucleotide variants. The present specification relates to a general pipeline for SV detection from long-read sequencing data. In particular, there is provided a method that encompasses a combination of alignment-based and assembly-based methods, enabling the detection of both simple SVs (insertions, deletions, inversions, and duplications) and potential complex SVs formed by combinations of simple SVs.

Through extensive simulation studies, it can be shown that embodiments of the invention can achieve superior performance compared with existing tools in SV detection. Notably, embodiments of the invention excel in complex SV detection. Furthermore, embodiments of the invention can be applied along with other tools, to the HG002 dataset. Embodiments of the invention not only perform well in detecting benchmark insertions and deletions, but also identifies numerous potential complex SVs of interest.

The present specification relates to a general SV detector that uses third-generation sequencing data. The pipeline comprises one or more of the following six steps: encoding, detecting, clustering, assembling, aligning and finalizing:
Encoding: this step includes extracting SV signals from aligned reads in binary alignment map (BAM) files, utilizing both intra- and inter-alignment results.
Detecting: this step includes detecting regions with strong signal accumulation from reads using a graph cut method. For the sake of conciseness, details on the graph cut method will not be described in this specification. Further details can be obtained from: Zhou, X., Yang, C., Yu, W. Moving object detection by detecting contiguous outliers in the low-rank representation[J]. IEEE Transactions on Pattern Analysis and Machine Intelligence, 2013, 35(3):597-610.
Clustering: in this step, within each candidate region, the corresponding reads are clustered to account for potential different subtypes.
Assembling: in this step, reads within a cluster are assembled into a contig.
Aligning: in this step, the maximum exact match method is used for keeping the variant information as much as possible.
Finalizing: in this step, the above alignment results are synthesized to finalize the SV callings.

Embodiments of the invention combine the advantages of alignment-based and assembly-based methods and allow for the detection of complex SVs by deferring the assignment of SV type labels until the final alignment step. To evaluate the performance of embodiments, simulations were conducted and HG002 dataset are utilized, demonstrating its superiority over state-of-the-art tools. For the sake of conciseness, details on the HG002 dataset will not be described in this specification. Further details can be obtained from: Liao, W., Asri, M., Ebler, J. et al. A draft human pangenome reference[J]. Nature, 2023, 617: 312-324. Additionally, embodiments can be applied to detect SVs in breast cancer data. By leveraging the benefits of alignment-based and assembly-based methods, embodiments surpass existing tools in terms of recall, precision and F1-score. The ability to identify complex SVs further enhances its utility.

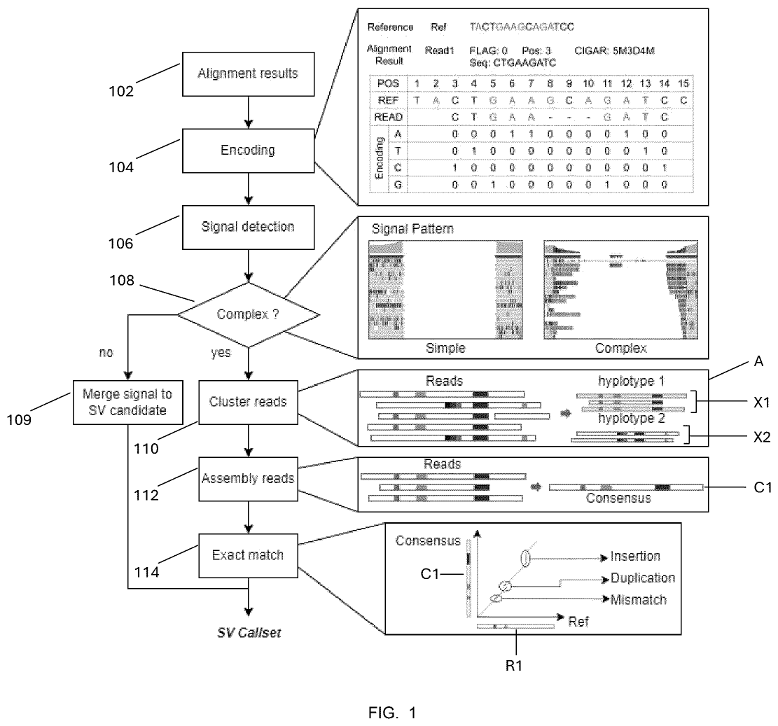

Each step of the pipeline plays a distinct role in the process and is explained in detail below with reference to FIG. 1. FIG. 1 is a flow chart illustrating a method for detecting structural variants (SVs) from long-read sequencing data, according to an embodiment.

### 1. Encoding (Step 104):

To identify as many SVs as possible, embodiments of the invention seek to retain as much information as possible during the early stages of detection. Unlike existing alignment-based SV detection tools, no predefined SV types are assigned at the beginning. Instead, inconsistencies between the reads and the reference sequence are first identified. These inconsistencies encompass sequencing errors and potential variant signatures.

A matrix-based approach is used to capture all inconsistencies. The reference sequence for each chromosome is encoded as a 4×reference length matrix. The alignment results (step 102) of each read are encoded as a 4×reference length matrix. The start and end positions of each result are also recorded to determine their positions in the reference. The differences between these matrices, obtained through subtraction, shows all the inconsistencies between the sample and the reference.

The sequence and specific match results of the read are stored in the sequence (SEQ) and CIGAR columns of the alignment file, respectively. For the CIGAR information, the alignment results typically include four options: “M” (match or mismatch), “I” (insertion), “D” (deletion), and “S” (soft-clipping). Soft clipping indicates no match, and these bases are skipped. For example, as shown in FIG. 1, the reference sequence is “TACTGAAGCAGATCC” and the read is “CTGAAGATC”. The captured CIGAR information from the alignment result is “5M3D4M”.

Different flags in the alignment results require different processing. A read with FLAG=“0” or “16” (indicates a single consecutive alignment result) is encoded. When the flag of an alignment result is “2048” (supplementary) or “2064” (2048+16, supplementary+reverse complemented), it implies that different parts of the read are aligned to different positions of the reference. In such cases, the read must also have an alignment result with FLAG=“0” or “16” (primary result). Referring back to FIG. 1, the FLAG=“0”. Hence, the single consecutive alignment result is encoded.

To reflect this positional relationship in the matrix, embodiments of the invention seek to encode (step 104) the primary and supplementary results in the same matrix. If they have the same direction, they are placed in the matrix based on their relative position relationship, with the region between them represented by 0. If the strands of the primary and supplementary results are different, the supplements are converted to their reverse complement to reflect this difference.

Compared to other prior art SV callers, the matrix-based encoding method according to embodiments of the invention preserves all inconsistencies, thus increasing the possibility of detecting more SVs and complex SVs. Let Sⱼ={0,1}represent whether the j-th position corresponding to the reference is a variant or not, rᵢⱼ=(aᵢⱼ, tᵢⱼ, cᵢⱼ, gᵢⱼ) indicates the encoding result for the j-th position in the i-th read, and refⱼ indicates the encoding result for the j-th position in the reference.

### 2. Detecting (Step 106):

After encoding (step 104) the signals in a matrix form, embodiments of the invention seek to detect candidate regions with strong SV signals. Using the above defined notations, the problem is formulated as follows:

$${min}_{{S}_{j} ∈ \{ 0 , 1 \}} {\sum }_{ij : {S}_{j} = 0} {‖ {r}_{ij} - {ref}_{j} ‖}^{2} + β {\sum }_{j} {S}_{j} + γ {\sum }_{( j , k ) ∈ ε} \vert {S}_{j} - {S}_{k} \vert .$$

The formula above comprises three parts. The first part represents the loss when a position is labelled as non-variant (Sⱼ=0), and the L₂ norm is used to quantify the inconsistency between reads and the reference. The second part serves as a penalty when a position is labelled as variant (Sⱼ=1), with the parameter β acting as a threshold for supporting evidence. The model trades off between labels 1 and 0 based on the first two terms. The third part is a penalty term penalizing different neighboring labels. The purpose is to further smooth the label sequence and encourage the detection of continuous variants. This formulation enables the identification of candidate regions with strong signals for further analysis.

### 3. Clustering:

In each candidate region, there may be multiple reads that belong to different subtypes, representing different SVs or genotypes within the region. If a user chooses to utilize the prior information obtained during the encoding step to help in the detection, the candidate regions can be classified into two categories: regions with distinct signatures and regions with complex, confusing signatures (usually containing complex SVs and some simple SVs). See step 108 at which the workflow is different for regions with distinct signatures (step 109) and regions with complex, confusing signatures (step 110).

For regions with distinct signatures, frequency-based clustering is used to determine the subtype or genotype of SVs within them. Specifically, embodiments of the invention cluster the signatures within regions based on starting position, length, and type. Signatures with the same type and similar length and position are grouped together. By analyzing the number of supporting reads, embodiments of the invention can determine their genotypes and then make a final call of their corresponding SV. This is shown at step 109.

For regions with complex and messy signatures, the alignment results are often too chaotic to discern what is happening in these regions. Therefore, embodiments of the invention use assembly and alignment techniques to determine the sequence structure. However, directly assembling reads corresponding to different subtypes together may lead to problematic contig that cannot reflect the feature for any subtype.

To address this problem, a clustering step (step 110) is introduced in each region. Given that SVs usually cover many bases, if multiple subtypes of SVs exist, the reads corresponding to different subtypes exhibit significant deviations within the region. However, if there is only one subtype, the differences among reads are mainly due to other factors such as sequencing errors or mapping errors. Importantly, embodiments of the invention allow only one subtype in one region, which is usually the case. This task differs somewhat from a classical clustering problem but fits within the framework of a statistical hypothesis test. The null hypothesis is that a pair of reads are from one subtype versus the alternative hypothesis that they are not from one subtype.

To construct a test statistic, the differences between each pair of reads are directly calculated. These differences are then compared against a user-defined threshold for screening purposes. The testing process is performed in the order of read length, from the longest read to the shortest, ensuring each read is typically examined only once. As a result, this algorithm runs efficiently with a time complexity of O(n), where n is the number of reads in the candidate region.

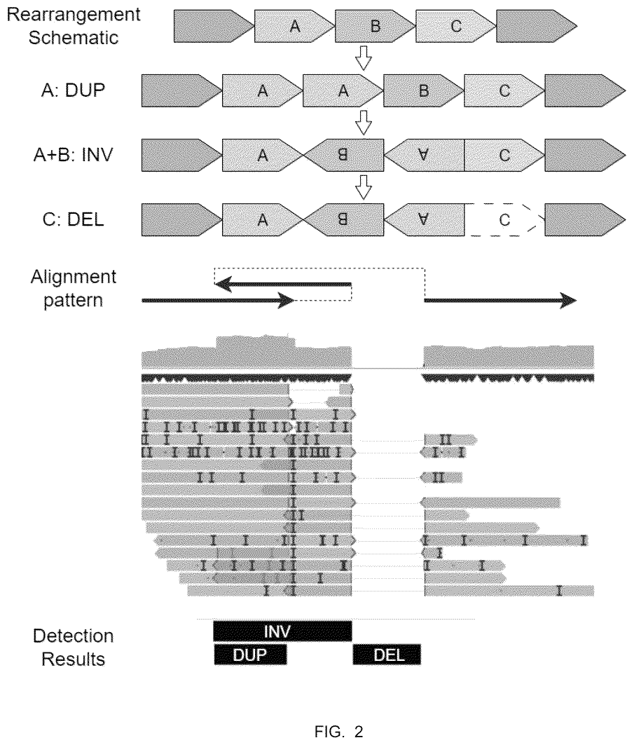

FIG. 2 shows an example of a simulated complex SV comprising deletion, duplication and inversion, where A part is duplicated first, then the duplicated A part and B part are inversed, and finally C part is deleted. Such complex SV with different types of simple SVs occurring together, is difficult to accurately detect by the existing tools. Embodiments of the invention can provide accurate callings presented at the bottom of the figure.

### 4. Assembling (Step 112):

For reads within a cluster, assembly tool wtdbg2 is used to generate a contig. For the sake of conciseness, details on assembly tool wtdbg2 will not be described in this specification. Further details can be obtained from: Ruan, J., Li, H. Fast and accurate long-read assembly with wtdbg2[J]. Nature Methods, 2020, 17: 155-158. This assembling step enables a user to obtain a longer contiguous sequence that represents the combined information from the reads within the cluster. The wtdbg2 tool is also used in Debreak (Chen, Y., Wang, A. Y., Barkley, C.A., et al. Deciphering the exact breakpoints of structural variations using long sequencing reads with debreak[J]. Nature Communications, 2023, 14(1): 283.) to detect long insertions by assembling the reads around them.

### 5. Aligning (Step 114):

With the assembled contig, it is aligned back to the reference genome to determine the variants present within the cluster. In embodiments of the invention, there is typically only one contig for each cluster potentially containing SV(s).

A maximum exact match (MEM) approach is used for the alignment step. For the sake of conciseness, details on MEM will not be described in this specification. Further details can be obtained from: Grabowski, S., Bieniecki, W. copMEM2: robust and scalable maximum exact match finding[J]. Bioinformatics, 2023, 39(5): btad313. MEM identifies and reports the maximum exact matches between the contig and the reference while disregarding some gaps. This approach, despite yielding some gaps, keeps as much information as possible and allows users to infer the differences between the contig and the reference more accurately. Moreover, MEM also enables users to capture information about potential complex SVs, if present. According to an implementation, the copmem2 tool is utilized to carry out the MEM alignment. For the sake of conciseness, details on the copmem2 tool will not be described in this specification. Further details can be obtained from: Grabowski, S., Bieniecki, W. copMEM2: robust and scalable maximum exact match finding[J]. Bioinformatics, 2023, 39(5): btad313.

### 6. Finalizing:

The alignment results obtained using MEMs are transformed into the final SV callings (see “SV Callset” in FIG. 1). This step is similar to the encoding step, but with additional considerations for inversions and gaps. When using MEMs, both forward matches and inversion matches are reported, along with their positions in the contig and reference sequences, as well as the match lengths. However, due to various factors such as sequencing errors, mapping errors, Single Nucleotide Polymorphism (SNP)s, and small indels, there may be gaps present in the alignments, apart from the SVs. These gaps are typically short, covering only a few base pairs (bps), but they can introduce gaps in both the contig and reference sequences. The difference in gap lengths between the contig and reference sequences is generally less than 50 base pairs. In embodiments of the invention, gaps not introduced by SVs are filled, and inversions are converted to the forward sequence. Subsequently, final SVs are determined based on the aforementioned encoding step.

Final SVs may not necessarily fall into common SV types such as deletions and insertions. Complex SVs, which involve combinations of common SVs, can be directly inferred from the MEM results. For potential complex SVs, the order of combinations of simple SVs matters a lot, which can also be inferred from MEMs.

In summary, embodiments of the invention relate to a general pipeline for SV detection from long-read sequencing data and encompasses a combination of alignment-based and assembly-based methods, enabling the detection of both simple SVs (insertions, deletions, inversions, and duplications) and complex SVs formed by combinations of simple SVs. SV signals are extracted from aligned reads in BAM files for encoding purposes, utilizing both intra- and inter-alignment results. Next, regions with strong signal accumulation are detected from reads using a graph cut method. Within each candidate region, the corresponding reads are clustered to account for potential different subtypes. Reads within a cluster are then assembled into a contig, which is mapped to the reference genome via maximum exact match.

To evaluate the performance of embodiments of the invention, simulations were conducted and HG002 dataset are utilized, demonstrating its superiority over state-of-the-art tools. Additionally, embodiments can be applied to detect SVs in breast cancer data. FIG. 3 is a table illustrating the performance of embodiments of the invention in detecting the simple SVs. By leveraging the benefits of alignment-based and assembly-based methods, embodiments (see row labelled “Ours”) surpass existing tools (such as SVIM, Sniffles, PBSV, CuteSV, Debreak) in terms of recall, precision and F1-score. The ability to identify complex SVs further enhances its utility.

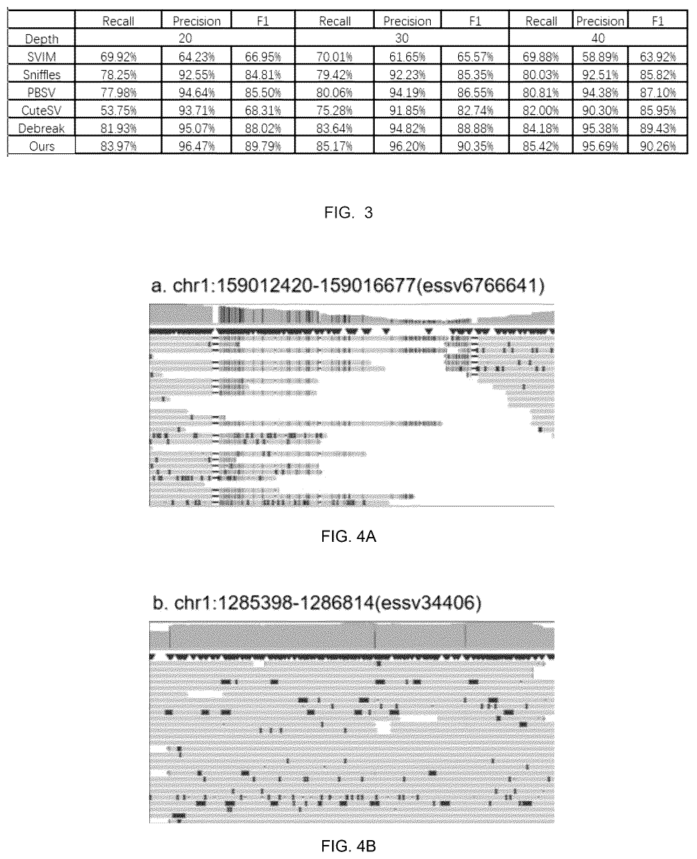

FIG. 4A is an Integrative Genomics Viewer (IGV) screenshot of a simulated DEL only detected by embodiments of the invention. FIG. 4B is a IGV screenshot of a simulated DUP only detected by embodiments of the invention. Note that the content within parentheses represents its identifier in the Database of Genomic Variants (DGV).

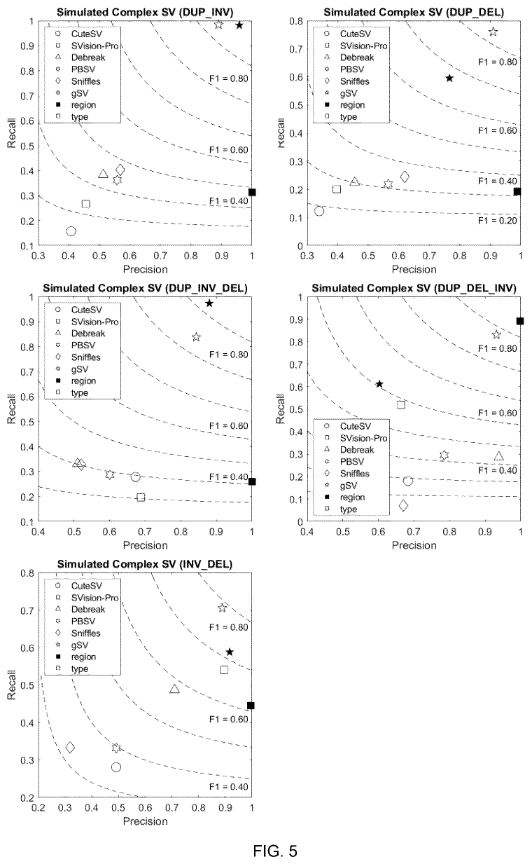

FIG. 5 shows the performance of candidate methods in detecting different types of complex SVs by evaluating the region and type respectively.

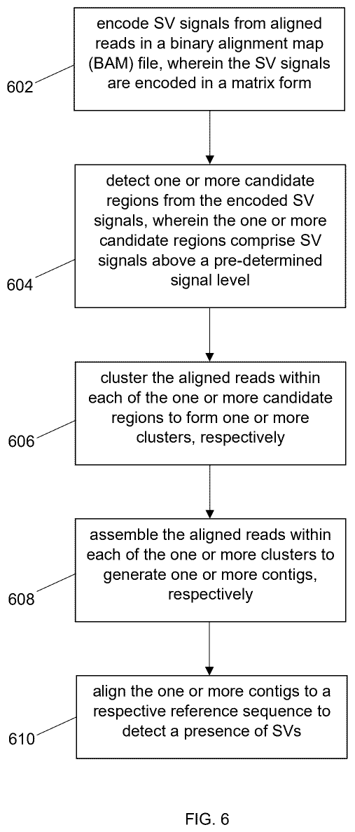

FIG. 6 is a flow chart illustrating a method for detecting structural variants (SVs) from long-read sequencing data. The method includes the step 602 of encoding SV signals from aligned reads in a binary alignment map (BAM) file, wherein the SV signals are encoded in a matrix form. Step 604 involves detecting one or more candidate regions from the encoded SV signals, wherein the one or more candidate regions comprise SV signals above a pre-determined signal level.

Step 606 involves clustering the aligned reads within each of the one or more candidate regions to form one or more clusters, respectively. There can be more than one candidate region. As an example, assume that there are two candidate regions: A and B. Refer to reference sign “A” in FIG. 1. Note that “B” is not shown in FIG. 1 for the sake of brevity and conciseness. There may be multiple aligned reads in each candidate region. For example, region A has five aligned reads. The five aligned reads in region A are clustered together to form cluster X1 and X2. See “X1” and “X2” in FIG. 1. There may be different types of subtypes in the same region, also known as hyplotypes. For any particular region, the reads are clustered so that they can correctly correspond to their own hyplotypes.

Step 608 involves assembling the aligned reads within each of the one or more clusters to generate one or more contigs, respectively. In cluster X1, the three aligned reads are assembled to form contig C1. The two aligned reads in X2 are not shown in FIG. 1 for the sake of brevity and conciseness.

Step 610 involves aligning the one or more contigs to a respective reference sequence to detect a presence of SVs. Contig C1 is aligned to reference sequence R1. Generally speaking, there is only one contig. In the event that there are multiple contigs, each contig may have its own unique reference sequence. In the end, SVs can be detected by comparing C1 with R1.

The step of aligning the one or more contigs to a respective reference sequence to detect a presence of SVs may include using copmem2 to perform maximum exact match (MEM) alignment.

Subsequent to aligning the one or more contigs to a respective reference sequence to detect a presence of SVs, the method further include filtering results of the MEM alignment to generate a SV calling. The filtering is based on inversions and/or gaps in the one or more contigs.

A wtdbg2 assembly tool can be used to assemble the aligned reads within each of the one or more clusters to generate the one or more contigs, respectively.

A graph cut method can be used for detecting the one or more candidate regions from the encoded SV signals.

According to an embodiment of the invention, there is provided a method for detecting structural variants (SVs) from long-read sequencing data, including one or more of the steps of: (a) encoding SV signals from aligned reads in BAM files, capturing inconsistencies between the reads and the reference genome; (b) detecting candidate regions with strong SV signals by minimizing the loss of non-variant positions and penalizing variant positions; (c) clustering reads within each candidate region to determine subtypes or genotypes of SVs; (d) assembling reads within each cluster into a contig using an assembly tool; (e) aligning the assembled contig to the reference genome using a maximum exact match method to identify variants present within the cluster; (f) finalizing the SV callings by transforming the alignment results into the final SV callings, considering inversions and gaps, and determining the final SV types based on the encoding step.

The encoding step may involve encoding SV signals in a matrix form, representing inconsistencies between the reads and the reference genome, and subtracting the matrices to obtain all the inconsistencies between the sample and the reference.

The detecting step may involve formulating a mathematical optimization problem to minimize the loss of non-variant positions, penalize variant positions, and penalize different neighboring labels, in order to detect candidate regions with strong SV signals.

The aligning step may involve using a maximum exact match method to align the assembled contig to the reference genome, capturing maximum exact matches and disregarding some gaps to accurately infer differences between the contig and the reference genome.

The finalizing step may involve transforming the alignment results obtained using maximum exact matches into final SV callings.

According to an embodiment of the invention, there is provided a system for detecting structural variants (SVs) from long-read sequencing data, including: a processor module; and a memory module including computer program code. The memory module and the computer program code are configured to, with the processor module, cause the system at least to: encode SV signals from aligned reads in a binary alignment map (BAM) file, wherein the SV signals are encoded in a matrix form; detect one or more candidate regions from the encoded SV signals, wherein the one or more candidate regions comprise SV signals above a pre-determined signal level; cluster the aligned reads within each of the one or more candidate regions to form one or more clusters, respectively; assemble the aligned reads within each of the one or more clusters to generate one or more contigs, respectively; align the one or more contigs to a respective reference sequence to detect a presence of SVs.

The system can be further caused to use copmem2 to perform maximum exact match (MEM) alignment for aligning the one or more contigs.

The system can be further caused to filter results of the MEM alignment to generate a SV calling, wherein the filtering is based on inversions and/or gaps in the one or more contigs.

The system can be further caused to use a wtdbg2 assembly tool to assemble the aligned reads within each of the one or more clusters to generate the one or more contigs, respectively.

The system can be further caused to use a graph cut method for detecting the one or more candidate regions from the encoded SV signals.

The system can be further caused to execute/perform other methods/steps described herein.

According to an embodiment, there is provided a computer-implemented method for detecting structural variants (SVs) from long-read sequencing data, the method comprising:

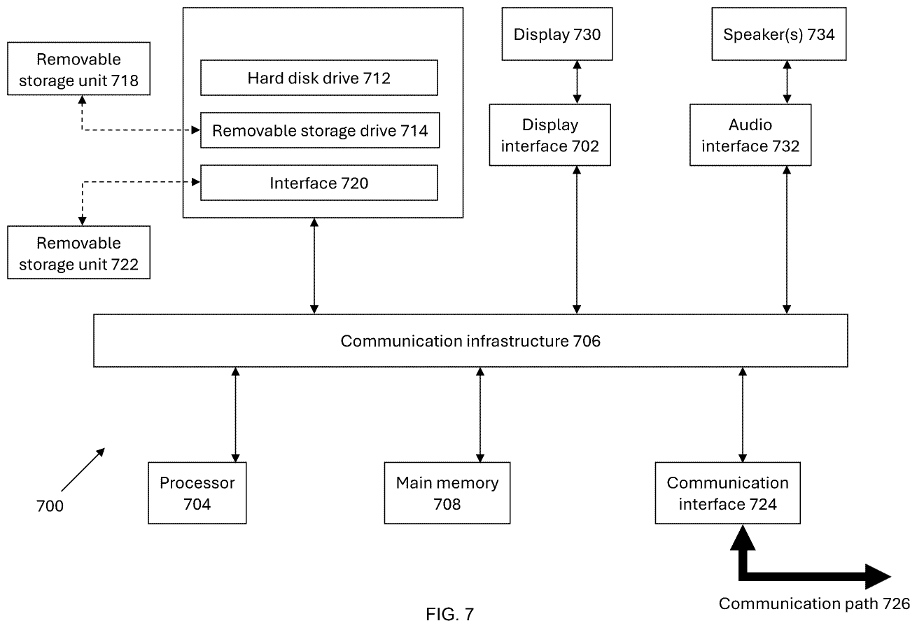

FIG. 7 shows a schematic diagram of a computer system suitable for use in executing at least some steps of the method for detecting structural variants (SVs) from long-read sequencing data.

FIG. 7 depicts an exemplary computing device 700, hereinafter interchangeably referred to as a computer system 700, where one or more such computing devices 700 may be to execute/perform methods for detecting structural variants, and/or other methods/steps described herein. The following description of the computing device 700 is provided by way of example only and is not intended to be limiting.

As shown in FIG. 7, the example computing device 700 includes a processor 704 for executing software routines. Although a single processor is shown for the sake of clarity, the computing device 700 may also include a multi-processor system. The processor 704 is connected to a communication infrastructure 706 for communication with other components of the computing device 700. The communication infrastructure 706 may include, for example, a communications bus, cross-bar, or network.

The computing device 700 further includes a main memory 708, such as a random access memory (RAM), and a secondary memory 710. The secondary memory 710 may include, for example, a hard disk drive 712 and/or a removable storage drive 714, which may include a floppy disk drive, a magnetic tape drive, an optical disk drive, or the like. The removable storage drive 714 reads from and/or writes to a removable storage unit 718 in a well-known manner. The removable storage unit 718 may include a floppy disk, magnetic tape, optical disk, or the like, which is read by and written to by removable storage drive 714. As will be appreciated by persons skilled in the relevant art(s), the removable storage unit 718 includes a computer readable storage medium having stored therein computer executable program code instructions and/or data.

In an alternative implementation, the secondary memory 710 may additionally or alternatively include other similar means for allowing computer programs or other instructions to be loaded into the computing device 700. Such means can include, for example, a removable storage unit 722 and an interface 720. Examples of a removable storage unit 722 and interface 720 include a program cartridge and cartridge interface (such as that found in video game console devices), a removable memory chip (such as an EPROM or PROM) and associated socket, and other removable storage units 722 and interfaces 720 which allow software and data to be transferred from the removable storage unit 722 to the computer system 700.

The computing device 700 also includes at least one communication interface 724. The communication interface 724 allows software and data to be transferred between computing device 700 and external devices via a communication path 726. In various embodiments of the inventions, the communication interface 724 permits data to be transferred between the computing device 700 and a data communication network, such as a public data or private data communication network. The communication interface 724 may be used to exchange data between different computing devices 700 which such computing devices 700 form part an interconnected computer network. Examples of a communication interface 724 can include a modem, a network interface (such as an Ethernet card), a communication port, an antenna with associated circuitry and the like. The communication interface 724 may be wired or may be wireless. Software and data transferred via the communication interface 724 are in the form of signals which can be electronic, electromagnetic, optical or other signals capable of being received by communication interface 724. These signals are provided to the communication interface via the communication path 726.

As shown in FIG. 7, the computing device 700 further includes a display interface 702 which performs operations for rendering images to an associated display 730 and an audio interface 732 for performing operations for playing audio content via associated speaker(s) 734.

As used herein, the term “computer program product” may refer, in part, to removable storage unit 718, removable storage unit 722, a hard disk installed in hard disk drive 712, or a carrier wave carrying software over communication path 726 (wireless link or cable) to communication interface 724. Computer readable storage media refers to any non-transitory tangible storage medium that provides recorded instructions and/or data to the computing device 700 for execution and/or processing. Examples of such storage media include floppy disks, magnetic tape, CD-ROM, DVD, Blu-Ray™ Disc, a hard disk drive, a ROM or integrated circuit, USB memory, a magneto-optical disk, or a computer readable card such as a PCMCIA card and the like, whether or not such devices are internal or external of the computing device 700. Examples of transitory or non-tangible computer readable transmission media that may also participate in the provision of software, application programs, instructions and/or data to the computing device 700 include radio or infra-red transmission channels as well as a network connection to another computer or networked device, and the Internet or Intranets including e-mail transmissions and information recorded on Websites and the like.

The computer programs (also called computer program code) are stored in main memory 708 and/or secondary memory 710. Computer programs can also be received via the communication interface 724. Such computer programs, when executed, enable the computing device 700 to perform one or more features of embodiments discussed herein. In various embodiments, the computer programs, when executed, enable the processor 704 to perform features of the above-described embodiments. Accordingly, such computer programs represent controllers of the computer system 700.

Software may be stored in a computer program product and loaded into the computing device 700 using the removable storage drive 714, the hard disk drive 712, or the interface 720. Alternatively, the computer program product may be downloaded to the computer system 700 over the communications path 726. The software, when executed by the processor 704, causes the computing device 700 to perform functions of embodiments described herein.

It is to be understood that the embodiment of FIG. 7 is presented merely by way of example. Therefore, in some embodiments one or more features of the computing device 700 may be omitted. Also, in some embodiments, one or more features of the computing device 700 may be combined together. Additionally, in some embodiments, one or more features of the computing device 700 may be split into one or more component parts.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

What is claimed is:

1. A method for detecting structural variants (SVs) from long-read sequencing data, comprising:
encoding SV signals from aligned reads in a binary alignment map (BAM) file, wherein the SV signals are encoded in a matrix form;
detecting one or more candidate regions from the encoded SV signals, wherein the one or more candidate regions comprise SV signals above a pre-determined signal level;
clustering the aligned reads within each of the one or more candidate regions to form one or more clusters, respectively;
assembling the aligned reads within each of the one or more clusters to generate one or more contigs, respectively;
aligning the one or more contigs to a respective reference sequence to detect a presence of SVs.

2. The method of claim 1, wherein the step of aligning the one or more contigs to a respective reference sequence to detect a presence of SVs comprises:
using copmem2 to perform maximum exact match (M E M) alignment.

3. The method of claim 2, wherein subsequent to aligning the one or more contigs to a respective reference sequence to detect a presence of SVs, the method further comprises:
filtering results of the ME M alignment to generate a SV calling, wherein the filtering is based on inversions and/or gaps in the one or more contigs.

4. The method of claim 1, comprising using a wtdbg2 assembly tool to assemble the aligned reads within each of the one or more clusters to generate the one or more contigs, respectively.

5. The method of claim 1, comprising using a graph cut method for detecting the one or more candidate regions from the encoded SV signals.

6. A system for detecting structural variants (SVs) from long-read sequencing data, comprising:
a processor module; and
a memory module including computer program code;
the memory module and the computer program code configured to, with the processor module, cause the system at least to:
encode SV signals from aligned reads in a binary alignment map (BAM) file, wherein the SV signals are encoded in a matrix form;
detect one or more candidate regions from the encoded SV signals, wherein the one or more candidate regions comprise SV signals above a pre-determined signal level;
cluster the aligned reads within each of the one or more candidate regions to form one or more clusters, respectively;
assemble the aligned reads within each of the one or more clusters to generate one or more contigs, respectively;
align the one or more contigs to a respective reference sequence to detect a presence of SVs.

7. The system of claim 6, wherein the system is further caused to use copmem2 to perform maximum exact match (MEM) alignment for aligning the one or more contigs.

8. The system of claim 7, wherein the system is further caused to filter results of the MEM alignment to generate a SV calling, wherein the filtering is based on inversions and/or gaps in the one or more contigs.

9. The system of claim 6, wherein the system is further caused to use a wtdbg2 assembly tool to assemble the aligned reads within each of the one or more clusters to generate the one or more contigs, respectively.

10. The system of claim 6, wherein the system is further caused to use a graph cut method for detecting the one or more candidate regions from the encoded SV signals.
